(19) United States
(12) Patent Application Publication
(10) Pub. No.: US 2026/0007578 A1
(43) Pub. Date: Jan. 8, 2026

(54) A SMART ORGANIZER SYSTEM

(71) Applicant: VOITHY TECH LTD., Abu Dhabi (AE)

(72) Inventor: Mohamed Osama Mohamed ORIKAT, Abu Dhabi (AE)

(73) Assignee: VOITHY TECH LTD., Abu Dhabi (AE)

(21) Appl. No.: 19/112,002

(22) PCT Filed: Aug. 1, 2023
(86) PCT No.: PCT/IB2023/057792

§ 371 Date: Mar. 14, 2025

(30) Foreign Application Priority Data
Sep. 19, 2022 (AE) P6001910/2022

(51) Int. Cl. A61J 7/04

(52) U.S. Cl. CPC A61J 7/0418; A61J 7/0427; A61J 7/0454

(57) ABSTRACT

A smart organizer system comprising containers, each container adapted to store, instruct consumption and/or dispense respective medicine/medical consumables/health supplements, one or more sensors adapted to determine one or more values associated with parameters related to a user, a micro controller in communication with the one or more sensors, configured to facilitate the one or more containers to consume or dispense medicines/medical consumables/health supplements based on a prescription, compare the one or more values with one or more predetermine values, determine a medical condition of the user based on the comparison, and facility the one or more containers to consume or dispense one or more medicines/medical consumables/health supplements to be consumed by the user, to treat the medical condition, a user interface configured to receive the prescriptions as input, provide details regarding consumption instructions, and provide live status of availability and condition, and a communication module.

### TECHNICAL FIELD

Embodiments of the present invention generally relate to medical technology and more particularly to a smart organizer system.

### BACKGROUND

Taking medicine as prescribed or medication adherence is important for controlling chronic conditions, treating temporary conditions, and overall long-term health and well-being. A personal connection with a health-care provider or pharmacist is an important part of medication adherence. Taking medicine on time, as prescribed, is essential to making sure your body has an effective amount of the drug at all times. If not, this can cause the disease to develop a resistance to the medicine or simply prolong the amount of time it takes to feel better. To get maximum benefit from your medications, it is important to take them exactly as prescribed by your doctor. In fact, chance of a better health outcome improves when one takes their medications as directed.

Drug dose frequency is called dosage regimen. Designing the correct dosage regimen is important for achieving the desired therapeutic efficacy and avoiding undesired effects. Because of significant homogeneity among humans, the dosage regimen is calculated on a population basis. Despite the same dose of drug, it produces variations in pharmacological response. Hence, every human body require a varied dosage of medicines and in varied interval.

Not taking medicines or health supplements on time or taking the them in inappropriate quantity can result in a myriad of side effects, deterioration of health condition, and/or development of stronger pathogens which can resist the medicines. This may extend and prolong usage of the medicines or health supplements which can come with harmful side effects. To provide medicines at regular interval, there are no sophisticated systems available. Available solutions include color coded containers containing various medicines. Many manual systems are available where medicines are taken based on a timer. However, these systems fail due to their human dependency. Due to which, there are times where either wrong medicine/supplements are taken by a patient, or are medicines are taken in wrong intervals or wrong doses.

Therefore, in light of the foregoing discussion, there exists a need to overcome the aforementioned drawbacks associated with timely and appropriate drug intake using a smart organizer system.

### SUMMARY OF THE INVENTION

According to an aspect of the present invention, there is provided a smart organizer system. The system comprises one or more containers, where each container of the one or more containers are adapted to store, instruct consumption, and/or dispense respective medicine, one or more sensors adapted to determine one or more values associated with parameters related to a user/patient and to the consumption of the stored medication, a micro controller in communication with the one or more sensors, comprising a memory unit configured to store machine-readable instructions, and a processor operably connected with the memory unit, the processor obtaining the machine-readable instructions from the memory unit, and being configured by the machine-readable instructions to facilitate the one or more containers to consume or dispense medicines/medical consumables/health supplements based on a prescription of medicines/medical consumables/health supplements, compare the one or more values with one or more predetermine values, determine a medical condition of the user/patient based on the comparison, and facility the one or more containers to consume or dispense one or more medicines/medical consumables/health supplements to be consumed by the user/patient, to treat the medical condition, determine a number of medicines/medical consumables/health supplements left in the respective one or more containers, a user interface, configured to receive the prescriptions as input for medicines/medical consumables/health supplements from any of concerned entities or the user/patient, and provide details regarding consumption instructions of the one or more medicines/medical consumables/health supplements to the user/patient, a communication module configured to send alerts related to the medical condition of the user/patient to entity devices held by the concerned entities, receive the prescriptions for medicines/medical consumables/health supplements from any of the concerned entities, and send a request placing an order at a supplier devices disposed at pharmacies for medicines/medical consumables/health supplements required for the user/patient.

In accordance with an embodiment of the present invention, the one or more sensors are disposed proximal to the one or more containers or on body of a user/patient.

In accordance with an embodiment of the present invention, the parameters are human health parameters or environment parameters.

In accordance with an embodiment of the present invention, the user interface is configured to provide live status of availability and condition of the medicines/medical consumables/health supplements inside the one or more containers.

In accordance with an embodiment of the present invention, the one or more containers have covers equipped with assistive tools for visually impaired users such as Braille dotted codes and/or vibrating latch.

In accordance with an embodiment of the present invention, the user interface is selected from a group comprising a display, an LED output, an audio output, a video output, and/or a vibrational output.

In accordance with an embodiment of the present invention, the one or more sensors are selected from a group comprising touch sensors, on/off sensors, heat sensors, temperature sensors, humidity sensors, infrared sensors, ultrasonic sensors, laser sensors, weight sensors, photo electric sensors, light sensors.

In accordance with an embodiment of the present invention, the system comprises a power source configured to provide power for operating the one or more sensors, the processor and the one or more containers.

In accordance with an embodiment of the present invention, the power source is a stationary and/or a rechargeable power source.

### BRIEF DESCRIPTION OF THE DRAWINGS

So that the manner in which the above recited features of the present invention can be understood in detail, a more particular description of the invention, briefly summarized above, may have been referred by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein

FIG. 1 illustrates a smart organizer system, in accordance with an embodiment of the present invention; and

FIGS. 2-4 illustrate an information flow diagram of the system, in accordance with an embodiment of the present invention.

### DETAILED DESCRIPTION OF THE DRAWINGS

The present invention is described hereinafter by various embodiments with reference to the accompanying drawing, wherein reference numerals used in the accompanying drawing correspond to the like elements throughout the description.

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e., meaning having the potential to), rather than the mandatory sense, (i.e., meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes.

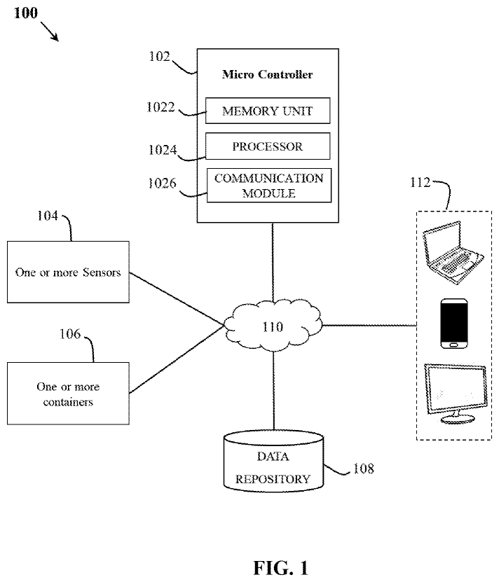

Referring to the drawings, the invention will now be described in more detail. FIG. 1 illustrates a smart organizer system 100, in accordance with an embodiment of the present invention. As shown in FIG. 1, the system 100 may comprise, but not limited to, one or more containers 106, one or more sensors 104, a communication module 1026, a user interface 112, and a micro controller 102.

As shown in FIG. 1, connected to the network 110 is the micro controller 102. The micro controller 102 may be a portable computing device, a desktop computer or a server stack. The micro controller 102 is envisaged to include means for computing capabilities, such as a memory unit 1022 configured to store machine readable instructions. The machine-readable instructions may be loaded into the memory unit 1022 from a non-transitory machine-readable medium such as, but not limited to, CD-ROMs, DVD-ROMs and Flash Drives. Alternately, the machine-readable instructions may be loaded in a form of a computer software program into the memory unit 1022. The memory unit 1022 in that manner may be selected from a group comprising EPROM, EEPROM and Flash memory. Further, the micro controller 102 includes a processor 1024 operably connected with the memory unit 1022. In various embodiments, the processor 1024 is one of, but not limited to, a general-purpose processor 1024, an application specific integrated circuit (ASIC) and a field-programmable gate array (FPGA).

Further, the user interface 112 may be connected with the network 110, are computing devices selected from a group comprising a laptop, mobile, a wearable watch or band, a desktop and a portable handheld device, having computing capabilities and comprising at least a processor 1024, a memory unit 1022, a display module, an input module and a user interface 112. In accordance with an embodiment of the present invention, the user interface 112 is selected from a group comprising a display, an LED output, an audio output, a video output, and/or a vibrational output. The user interface 112 may include a display envisaged to provide the video output in form of a visuals to show the data received from micro controller 102, the one or more sensors 104, the one or more containers 106, the communication module 1026, and the results of the processor 1024 analysis. The display may be, but not limited to, Light-emitting diode display (LED), electroluminescent display (ELD), liquid crystal display (LCD), Organic light-emitting diode (OLED) & AMOLED display electronic lights, video, electronic display. Furthermore, the user interface 112 may include accessories like keyboard, mouse etc. envisaged to provide input capability to enable the user/patients to enter his/her details mentioned above. In another embodiment, the user interface 112 may be a touch input-based display that integrates the input-output functionalities.

In accordance with an embodiment of the present invention, the one or more sensors 104 are connected with the micro controller 102. The one or more sensors 104 may be selected from a group comprising, but not limited to, touch based sensors, heat sensors, temperature sensors, humidity sensors, infrared sensors, ultrasonic sensors, laser sensors, weight sensors, photo electric sensors, pulse sensor, and light sensors. The one or more sensors 104 are disposed proximal to the one or more containers 106 or on body of a user/patient.

In accordance with an embodiment of the present invention, each container 106 of the one or more containers 106 adapted to store instruct consumption and/or dispense respective medicine/health supplements or medical consumables. The one or more containers 106 are automated containers 106 and may comprise motors, rachets, belts, or conveyers to dispense medicines/medical consumables/health supplements. The one or more containers 106 may be made of storage specific material to keep content in acceptable storage conditions while being stored in the compartment (example: medical grade material, thermal and humidity resistance, or shock resistance). Further the one or more containers 106 may comprise latch to cover the containers 106 and control access. The latch may be made from non-translucent material to prevent light from damaging sensitive medicines/medical consumables/health supplements. The one or more containers 106 may have latches/covers equipped with assistive tools for visually impaired users such as Braille dotted codes and/or vibrating latch. The one or more containers 106 may be connected with the micro controller 102 using the network 110. In accordance with an embodiment of the present invention, the medical consumables may include, but not limited to, gloves, syringes, bandages, cotton wool, sutures, catheters, tubes, infusion sets, IV cannulas etc. Laboratory supplies, including pipettes, blood collecting tubes and microscope supplies. Surgical instruments, such as scalpels, forceps, lancets, scissors, instrument boxes etc. Further, the health supplements may include vitamin, mineral capsules or pills such as zinc, magnesium, omega 3, phosphorous, etc. Further, the medicines may include, but not limited to, chemical compositions in pills, capsule, liquid, or any other form to cure a particular disease and/or maintain a healthy condition.

Further, the micro controller 102 comprises a communication module 1026 configured for enabling connection of micro controller 102, the user interface 112, entity devices 234, and supplier devices 232. The connection may be wired or wireless. In that sense, the communication module 1026 may include Power over Ethernet Switch, USB ports etc. These may allow transferring of data among the various components of the system 100 via Ethernet cable, USB cable etc. Additionally, or alternately, the communication module 1026 may be an Internet of Things IOT module, Wi-Fi module, Bluetooth module, RF module etc. adapted to enable a wireless communication between the user interface 112, entity devices, and the supplier devices, the processor 1024, the one or more sensors 104, and the one or more containers 106 via a wireless communication network 110.

In an additional or alternative embodiment, the system 100 may include one or more user devices, entity devices (not shown in FIG. 1) held by concerned entities, and supplier devices (not shown in FIG. 1) held by various pharmacies and may be connected with the micro controller 102 via a wired or wireless connection. Herein, the one or more user devices, the entity devices, and the supplier devices may be selected from computing devices such as desktop PC, laptop, PDA or hand-held computing device such as smartphones and tablets. The one or more user devices are associated with one or more users. In another embodiment, instead of a micro controller 102 being a stand-alone device, the one or more user devices, the entity devices, and the supplier devices may house the micro controller along with their functionalities. The one or more user device, the entity devices, and the supplier devices already includes microprocessor for processing and communication capabilities via wired or wireless connections.

In accordance with an additional or alternative embodiment of the present invention, the micro controller 102 may also include a registration module adapted to receive user/patient's, concerned entities' details and register them on the system 100. The details may include, but not limited to, username, contact number, email ID, age, gender, areas of interest, past history of illness etc. Besides, the registration module may also have a biometrics receiver configured to register the user/patient via his/her biometrics. The biometrics may include, fingerprint recognition, face recognition, Iris recognition or a combination thereof. In one embodiment, the micro controller 102 may be a stand-alone device where the user/patient may come and register himself/herself. Further, the registration module may be used to authenticate and authorize access to the user/patient. For example, a biometric, passcode, password, NFC, or proximity sensor or a combination of multiple authentication methods. Therefore, the system 100 can control and/or track the access to the stored content to ensure safety storage and authorized access.

In accordance with an embodiment of the present invention, the system 100 may further comprise a power source configured to provide power for operating the one or more sensors 104, the processor 1024, the communication module 1026, the user interface 112, and the one or more containers 106. The power source may a rechargeable power source such as lithium-Ion battery. In accordance with an embodiment of the present invention, the rechargeable power source may be charged using solar energy. The system may use solar cells/panels or wireless charging to charge the power source or power the system. In accordance with an additional or alternative embodiment, the power source may be a constant power source such as electricity from, but not limited to, wall plug car plug terminals. Also, other means of power sources may be connected or embedded in the device.

In accordance with an embodiment of the present invention, as shown in FIG. 1, the system 100 may also include a data repository 108. The data repository 108 may be a local storage (such as SSD, eMMC, Flash, SD card, etc.) or a cloud-based storage. In any manner, the data repository 108 is envisaged to be capable of providing the data to the micro controller 102, when the data is queried appropriately using applicable security and other data transfer protocols. The data repository 108 may store, but not limited to, texts, images, videos, audios, and 3D immersive content. It is also envisaged to store various charts, tables, learning contents such as practical videos, manipulatable 3D content, prepared for the users. In one embodiment of the present invention, the micro controller 102 may include AI and deep learning-based trained models using the above data, to compare and assess and update the database based on the received data from the sensors 104, the user interface 112, the one or more containers 106, the user devices, the supplier devices, the entity devices and/or the internet in real-time.

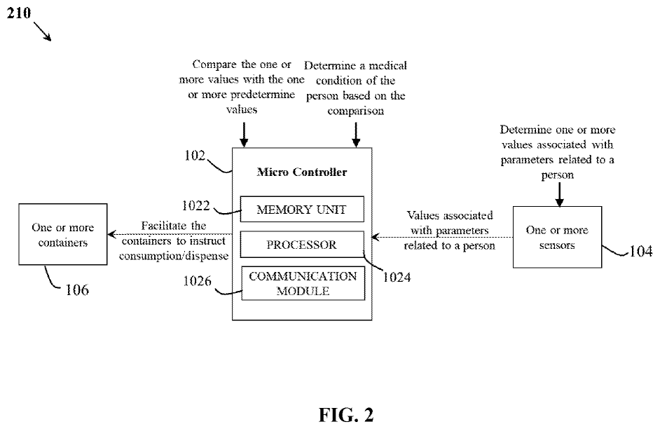

FIGS. 2-4 illustrate information flow diagram of the system 100, in accordance with an embodiment of the present invention. Firstly, as shown in FIG. 2, one or more sensors 104 adapted to determine one or more values associated with parameters related to a user/patient. The parameters are human health parameters and may be associated with any possible decease detectable using sensors. In accordance with an exemplary embodiment, the human health parameters may be selected from a group comprising, but not limited to weight, humidity, blood oxygen saturation, blood sugar, blood glucose, heart status, blood pressure, body temperature, breathing rate, heart rate, pulse rate, fluid balance, internal bleeding, skin parameters, respiratory parameters and various electrophysiological signals. Further, the human health parameters may include, parameters to detect obesity, arthritis, diabetes, asthma, cancer incidence, chronic pain, oral health, depression, hospital visits due to injury, reports of waterborne diseases or foodborne illness, anemia, etc. For example, consider the sensor as pulse sensor which is configured to determine a value of parameter such as heart rate as 92 beats per minute. Further, as shown in FIG. 2, the micro controller 102 in communication with the one or more sensors 104, configured to compare the one or more values with one or more predetermine values and determine a medical condition of the user/patient based on the comparison. For Example, normally, the predetermined value of the heart rate is around 80 beats per minute, therefore the micro controller 102 may compare the determined heart rate value i.e., 92 beats per minute with the predetermined value of the heart rate i.e., 80 beats per minute.

Since the determined value of the parameter, is higher than the predetermined value of the parameter, the micro controller 102 may determine a medical condition of the user/patient based on the comparison as may be, but not limited to, high blood pressure or anxiety. Further, as shown in FIG. 2, the micro controller 102 is configured to facility the one or more containers 106 to consume or dispense one or more medicines/medical consumables/health supplements to be consumed by the user/patient, to treat the medical condition. For example, on detection of the medical condition as high blood pressure, the micro controller 102 may treat the user/patient by dispensing an antihypertensive drug for the treatment. Further the user interface 112 is configured to provide details regarding consumption instructions of the one or more medicines/medical consumables/health supplements to the user/patient. The user interface 112, for example may provide a dosage and time duration of the dispensing drug for treatment. In alternative embodiment, the system 100 may be used with AI technology to assist users based on their behavior. In accordance with an alternative or additional embodiment, the system 100 may be able to communicate with other systems that might be needed to trigger or adjust consumption event of a medicine. For example, if the user is wearing a wearable device that detect a low blood sugar or irregular heart status, then the system 100 can use this information to guide the user to take or change the dose of a specified medicine. Further, the parameters may be environment parameters selected from, but not limited to, light, pressure, temperature, etc. The environment parameters may be related to an environment around the system and/or the human. The one or more sensors may also be adapted to determine the environment parameters.

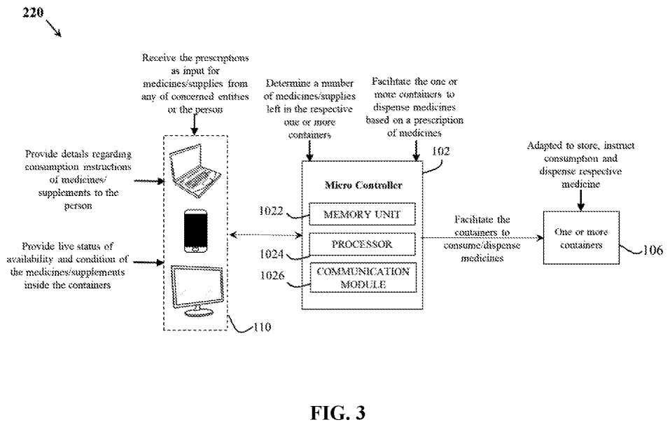

Further, as shown in FIG. 3, in accordance with an embodiment of the present invention, the user interface 112 is configured to receive a prescription for medicines/medical consumables/health supplements as input for medicines/medical consumables/health supplements from any of concerned entities such as caregivers, medical professional, health insurance companies, pharmacies, etc. or the user/patient. In accordance with an embodiment of the present invention, the user interface may include an integrated (embedded) medically approved computer program/algorithm and can be operated with authorized medical professionals. For example, a user/patient may be suffering from a viral fever, therefore, the medical professional may input the prescription for medicines/medical consumables/health supplements into the user interface 112. In an embodiment of the present invention, the communication module 1026 is configured to receive the prescriptions for medicines/medical consumables/health supplements from any of the concerned entities. For example, in case, the medical professional is distant, the medical professional may enter the prescription via entity devices 234 using the communication module 1026. In accordance with an embodiment of the present invention, the system 100 may take into account smoking habits, alcohol consumption habits, physical exercise habits, breastfeeding, or any other environment as input to determine a medicine intake routine.

The micro controller 102 in communication with the one or more containers 106 is configured to facilitate the one or more containers 106 to consume or dispense medicines/medical consumables/health supplements based on a prescription of medicines/medical consumables/health supplements. The micro controller 102 may enable the one or more containers 106 to instruct consumption and/or dispense the required medicines/medical consumables/health supplements periodically based on a schedule defined in the prescription. The micro controller 102 may reads schedule information and compare it with a real time clock information. For example, when schedule time is matched with the real time, the micro controller 102 operates the user interface such as audio output for an audible notification to the user. Further, the user interface provides container indicators for a notification determining which container's medicine/medical consumables/health supplements is to be taken and how many units of the medicines/medical consumables/health supplements to be consumed. Further, the user can use input button to mute audio output. When the user opens the container for medicine, the one or more sensors such as touch sensor will send information to micro controller 102 on timing and duration of the opening event until the latch on the container is closed. Further, the sensor may detect if the container is open, the duration of the container being open and/or close. Based on the detection, the micro controller may send alerts to the user/patient, the medical professionals, and any other interested entity. Further, the micro controller 102 updates data records to log consumption time and deduct stored quantity based on assumed consumption from schedule record.

In accordance with an embodiment of the present invention, the user interface 112 is configured to provide details regarding consumption instructions of the one or more medicines/medical consumables/health supplements to the user/patient. The prescription may prove a quantity of medicine/medical consumables/health supplements along with a schedule for intake or application. The system 100 should be able to store and play audible sounds and recordings using the user interface 112 to assist user in their medication regimen. Therefore, the system 100 can be used with embedded dispensing mechanism to alert consumption or automatically dispense the required item in the correct amount. Further, in accordance with an embodiment of the present invention, the micro controller 102 is configured to determine a number of medicines/medical consumables/health supplements left in the respective one or more containers 106. Furthermore, the user interface 112 is configured to provide live status of availability and condition of the medicines/medical consumables/health supplements inside the one or more containers 106. For example, the user interface 112 may determine availability of medicine/medical consumables/health supplements in the one or more containers 106. Further, the user interface 112 may provide status of expiry of medicines/medical consumables/health supplements and quantity of medicines/medical consumables/health supplements left in the one or more containers 106. For example, in case the container 106 is infected with molds, or the container 106 has been exposed to a level or duration more than a predetermined safe levels and/or predetermined safe duration of heat and/or humidity and/or light, or any other factor(s) which may contaminate medicines/medical consumables/health supplements, indicators of the user interface 112 may turn on indicating contamination of the containers 106 and medicines/medical consumables/health supplements.

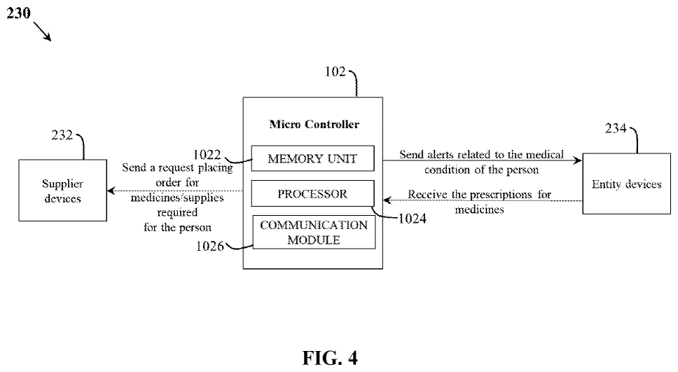

Furthermore, as shown in FIG. 4, the communication module 1026 is configured to receive the prescriptions for medicines/medical consumables/health supplements from any of the concerned entities using entity devices 234. In accordance with an embodiment of the present invention, the communication module 1026 is configured to send a request for placing an order at supplier devices 232 disposed at pharmacies for medicines/medical consumables/health supplements required for the user/patient. For example, in case, the one or more containers 106 are running out of medicines/medical consumables/health supplements for hypertension, the communication module 1026 may send a request for placing an order to supplier devices 232 placed at one or more warehouses having such medicines/medical consumables/health supplements for hypertension.

This way, the system 100 can be used with external systems to send re-stocking requests based on logged consumption and resulted low-stock levels. The system 100 can control and/or track the access to the stored content to ensure safety storage and authorized access. Furthermore, the communication module 1026 is configured to send alerts related to the medical condition of the user/patient to entity devices 234 held by the concerned entities. For example, the communication module 1026 may, on occurrence of hypertension, may send the alerts to the medical professionals regarding the occurrence of the medical condition. The system 100 can be used to in conjunction with other IoT and electronic systems to send real-time data to other systems and stakeholders. Example: in healthcare domain, the system 100 can send consumption timings of specific medicine, along with a health monitor reading that can show the doctor the effect of this medicine on the patient's conditions in real-time.

The system 100 can be used as part of organizational or enterprise systems to extend assistance and control levels. For example, in healthcare industry, the system 100 can be used with healthcare facilities to ensure proper adherence to medication either for in-patient and/or out-patient. The system 100 can be made into a portable device and can be provided to the user with pre-filled content and pre-programmed schedule. Once a low stock alert is received by the healthcare facility, another device will be dispatched and old one to be collected.

The invention has various advantages. The system automates the timely consumption of medicines/medical consumables/health supplements in accurate dosage. Proper consumption of medicines/medical consumables/health supplements reduce recovery time and improve health drastically. Further, in case of emergencies, such as hyper tension or stroke, the sensors provide a precautionary alert and suggest dosage which results in preventing major illness. The dose of medicines/health supplements may be suggested via a medically approved professional/microprocessor/electronic computer system/algorithm. Furthermore, the system can preorder medicines/medical consumables/health supplements so that regular supply of the medicines/medical consumables/health supplements can be kept.

The notifications provided by the system are touch, sound and visual based, hence, the user/patient cannot miss the notification for intake of the medicines/medical consumables/health supplements. Since all the logs of medicine/supplements intakes are saved, a medical professional can look into the logs and look into the patient's health to prescribe a new schedule and/or new set of medicines/medical consumables/health supplements and feed the same into the system. In this way, the current system can overcome the drawbacks of the existing solutions thereby providing technical advancement over the prior arts.

In general, the word “module,” as used herein, refers to logic embodied in hardware or firmware, or to a collection of software instructions, written in a programming language, such as, for example, Java, C, or assembly. One or more software instructions in the modules may be embedded in firmware, such as an EPROM. It will be appreciated that modules may comprised connected logic units, such as gates and flip-flops, and may comprise programmable units, such as programmable gate arrays or processors. The modules described herein may be implemented as either software and/or hardware modules and may be stored in any type of computer-readable medium or other computer storage device.

Further, while one or more operations have been described as being performed by or otherwise related to certain modules, devices or entities, the operations may be performed by or otherwise related to any module, device or entity. As such, any function or operation that has been described as being performed by a module could alternatively be performed by a different server, by the cloud computing platform, or a combination thereof. It is implied that the techniques of the present disclosure might be implemented using a variety of technologies. For example, the methods described herein may be implemented by a series of computer executable instructions residing on a suitable computer readable medium. Suitable computer readable media may include volatile (e.g., RAM) and/or non-volatile (e.g., ROM, disk) memory, carrier waves and transmission media. Exemplary carrier waves may take the form of electrical, electromagnetic or optical signals conveying digital data steams along a local network or a publicly accessible network such as the Internet.

Further, the operations need not be performed in the disclosed order, although in some examples, an order may be preferred. Also, not all functions need to be performed to achieve the desired advantages of the disclosed system and method, and therefore not all functions are required.

The terms and descriptions used herein are set forth by way of illustration only and are not meant as limitations. Examples and limitations disclosed herein are intended to be not limiting in any manner, and modifications may be made without departing from the spirit of the present disclosure. Those skilled in the art will recognize that many variations are possible within the spirit and scope of the disclosure, and their equivalents, in which all terms are to be understood in their broadest possible sense unless otherwise indicated.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claims.

1. A smart organizer system, the system comprising:
one or more containers, wherein each container of the one or more containers adapted to store and consume or dispense respective medicine;
one or more sensors adapted to determine one or more values associated with parameters related to a user/patient;
a micro controller in communication with the one or more sensors, comprising:
a memory unit configured to store machine-readable instructions; and
a processor operably connected with the memory unit, the processor obtaining the machine-readable instructions from the memory unit, and being configured by the machine-readable instructions to:
facilitate the one or more containers to consume or dispense medicines/medical consumables/health supplements based on a prescription;
compare the one or more values with one or more predetermine values;
determine a medical condition of the user/patient based on the comparison; and
facility the one or more containers to consume or dispense one or more medicines/medical consumables/health supplements to be consumed by the user/patient, to treat the medical condition;
determine a number of medicines/medical consumables/health supplements left in the respective one or more containers;
a user interface, configured to:
receive the prescriptions as input for medicines/medicines/medical consumables/health supplements from any of concerned entities or the user/patient; and
provide details regarding consumption instructions of the one or more medicines/medical consumables/health supplements to the user/patient;
a communication module configured to:
send alerts related to the medical condition of the user/patient to entity devices held by the concerned entities;
receive the prescriptions for medicines/medical consumables/health supplements from any of the concerned entities; and
send a request placing an order at supplier devices disposed at pharmacies for medicines/medical consumables/health supplements required for the user/patient.

2. The system as claimed in claim 1, wherein the one or more sensors are disposed proximal to the one or more containers or on body of a user/patient.

3. The system as claimed in claim 1, wherein the parameters are human health parameters.

4. The system as claimed in claim 1, wherein the user interface is configured to provide live status of availability and condition of the medicines/medical consumables/health supplements inside the one or more containers.

5. The system as claimed in claim 1, wherein the one or more containers have covers equipped with assistive tools for visually impaired users such as Braille dotted codes and/or vibrating latch.

6. The system as claimed in claim 1, wherein the user interface is selected from a group comprising a display, an LED output, an audio output, a video output, and/or a vibrational output.

7. The system as claimed in claim 1, wherein the one or more sensors are selected from a group comprising heat sensors, temperature sensors, humidity sensors, infrared sensors, ultrasonic sensors, laser sensors, weight sensors, photo electric sensors, light sensors.

8. The system as claimed in claim 1, comprising a power source configured to provide power for operating the one or more sensors, the processor and the one or more containers.

9. The system as claimed in claim 8, wherein the power source is a stationary and/or a rechargeable power source.
